(19) United States
(12) Patent Application Publication
(10) Pub. No.: US 2024/0246949 A1
(43) Pub. Date: Jul. 25, 2024

(54) SOLID STATE FORMS OF LANIFIBRANOR AND PROCESS FOR PREPARATION THEREOF

(71) Applicant: TEVA PHARMACEUTICALS INTERNATIONAL GMBH, Jona (CH)

(72) Inventors: Parven Kumar Luthra, Noida (IN); Anantha Rajmohan Muthusamy, Sivakasi (IN); Amit Singh, Greater Noida (IN)

(21) Appl. No.: 18/563,467

(22) PCT Filed: Jun. 10, 2022
(86) PCT No.: PCT/US2022/032967

§ 371 Date: Nov. 22, 2023

(30) Foreign Application Priority Data
Jun. 10, 2021 (IN) 202111025916
Apr. 28, 2022 (IN) 202211025057

(51) Int. Cl. C07D 417/12; A61K 31/428

(52) U.S. Cl. CPC C07D 417/12; A61K 31/428

(57) ABSTRACT

The present disclosure encompasses solid state forms of Lanifibranor, in embodiments crystalline polymorphs of Lanifibranor, processes for preparation thereof, and pharmaceutical compositions thereof.

### FIELD OF THE DISCLOSURE

The present disclosure encompasses solid state forms of Lanifibranor, in embodiments crystalline polymorphs of Lanifibranor, processes for preparation thereof, and pharmaceutical compositions thereof.

### BACKGROUND OF THE DISCLOSURE

Lanifibranor, 1-(6-benzothiazolylsulfonyl)-5-chloro-1H-indole-2-butanoic acid, has the following chemical structure:

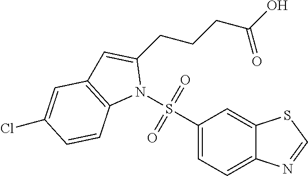 [SMILES: O=C(O)CCCc1cc2cc(Cl)ccc2n1S(=O)(=O)c1ccc2ncsc2c1]

Lanifibranor is a pan peroxisome proliferator-activated receptor, and it is developed for the treatment of non-alcoholic steatohepatitis (NASH), non-alcoholic fatty liver disease, type 2 diabetes, fibrosis and systemic sclerosis.

The compound is described in International Publication No. WO2007/026097.

Polymorphism, the occurrence of different crystalline forms, is a property of some molecules and molecular complexes. A single molecule may give rise to a variety of polymorphs having distinct crystal structures and physical properties like melting point, thermal behaviors (e.g., measured by thermogravimetric analysis (“TGA”), or differential scanning calorimetry (“DSC”)), X-ray diffraction (XRD) pattern, infrared absorption fingerprint, and solid state (¹³C) NMR spectrum. One or more of these techniques may be used to distinguish different polymorphic forms of a compound.

Different salts and solid state forms (including solvated forms) of an active pharmaceutical ingredient may possess different properties. Such variations in the properties of different salts and solid state forms and solvates may provide a basis for improving formulation, for example, by facilitating better processing or handling characteristics, changing the dissolution profile in a favorable direction, or improving stability (polymorph as well as chemical stability) and shelf-life. These variations in the properties of different salts and solid state forms may also offer improvements to the final dosage form, for instance, if they serve to improve bioavailability. Different salts and solid state forms and solvates of an active pharmaceutical ingredient may also give rise to a variety of polymorphs or crystalline forms, which may in turn provide additional opportunities to assess variations in the properties and characteristics of a solid active pharmaceutical ingredient.

Discovering new solid state forms and solvates of a pharmaceutical product may yield materials having desirable processing properties, such as ease of handling, ease of processing, storage stability, and ease of purification or as desirable intermediate crystal forms that facilitate conversion to other polymorphic forms. New solid state forms of a pharmaceutically useful compound can also provide an opportunity to improve the performance characteristics of a pharmaceutical product. It enlarges the repertoire of materials that a formulation scientist has available for formulation optimization, for example by providing a product with different properties, including a different crystal habit, higher crystallinity, or polymorphic stability, which may offer better processing or handling characteristics, improved dissolution profile, or improved shelf-life (chemical/physical stability). For at least these reasons, there is a need for additional solid state forms (including solvated forms) of Lanifibranor.

### SUMMARY OF THE DISCLOSURE

The present disclosure provides crystalline polymorphs of Lanifibranor, processes for preparation thereof, and pharmaceutical compositions thereof. These crystalline polymorphs can be used to prepare other solid state forms of Lanifibranor, Lanifibranor salts and their solid state forms.

In embodiments, the present disclosure provides Lanifibranor and crystalline forms thereof. In embodiments, the present disclosure provides crystalline forms of Lanifibranor designated as Form LN1 and Form LN2 (defined herein).

The present disclosure also provides uses of the said solid state forms of Lanifibranor in the preparation of other solid state forms of Lanifibranor or salts thereof.

The present disclosure provides crystalline polymorphs of Lanifibranor for use in medicine, including for the treatment of non-alcoholic steatohepatitis (NASH), non-alcoholic fatty liver disease, type 2 diabetes, fibrosis and systemic sclerosis.

The present disclosure also encompasses the use of crystalline polymorphs of Lanifibranor of the present disclosure for the preparation of pharmaceutical compositions and/or formulations.

In another aspect, the present disclosure provides pharmaceutical compositions comprising crystalline polymorphs of Lanifibranor according to the present disclosure.

The present disclosure includes processes for preparing the above mentioned pharmaceutical compositions. The processes includes combining any one or a combination of the crystalline polymorphs of Lanifibranor with at least one pharmaceutically acceptable excipient.

The crystalline polymorph of Lanifibranor as defined herein and the pharmaceutical compositions or formulations of the crystalline polymorph of Lanifibranor may be used as medicaments, such as for the treatment of non-alcoholic steatohepatitis (NASH), non-alcoholic fatty liver disease, type 2 diabetes, fibrosis and systemic sclerosis.

The present disclosure also provides methods of treating non-alcoholic steatohepatitis (NASH), non-alcoholic fatty liver disease, type 2 diabetes, fibrosis and systemic sclerosis, by administering a therapeutically effective amount of any one or a combination of the crystalline polymorphs of Lanifibranor of the present disclosure, or at least one of the above pharmaceutical compositions, to a subject suffering from non-alcoholic steatohepatitis (NASH), non-alcoholic fatty liver disease, type 2 diabetes, fibrosis and systemic sclerosis, or otherwise in need of the treatment.

The present disclosure also provides uses of crystalline polymorphs of Lanifibranor of the present disclosure, or at least one of the above pharmaceutical compositions, for the manufacture of medicaments for treating e.g. non-alcoholic steatohepatitis (NASH), non-alcoholic fatty liver disease, type 2 diabetes, fibrosis and systemic sclerosis.

### BRIEF DESCRIPTION OF THE DRAWINGS

FIG. 1 shows a characteristic X-ray powder diffraction pattern (XRPD) of Lanifibranor Form LN1.

FIG. 2 shows a characteristic XRPD of Lanifibranor Form LN2.

FIG. 3 shows a characteristic XRPD of Lanifibranor amorphous form.

FIG. 4 shows a characteristic XRPD of Lanifibranor Form LN3.

FIG. 5 shows a characteristic XRPD of Lanifibranor Form LN4.

FIG. 6a shows ¹³C solid state NMR spectrum of Form LN1 of Lanifibranor (full scan).

FIG. 6b shows ¹³C solid state NMR spectrum of Form LN1 of Lanifibranor (at the range of 0-100 ppm).

FIG. 6c shows ¹³C solid state NMR spectrum of Form LN1 of Lanifibranor (at the range of 100-200 ppm).

FIG. 7a shows ¹³C solid state NMR spectrum of Form LN2 of Lanifibranor (full scan).

FIG. 7b shows ¹³C solid state NMR spectrum of Form LN2 of Lanifibranor (at the range of 0-100 ppm).

FIG. 7c shows ¹³C solid state NMR spectrum of Form LN2 of Lanifibranor (at the range of 100-200 ppm).

### DETAILED DESCRIPTION OF THE DISCLOSURE

The present disclosure encompasses solid state forms of Lanifibranor, including crystalline polymorphs of Lanifibranor, processes for preparation thereof, and pharmaceutical compositions thereof.

In embodiments, the present disclosure provides Lanifibranor and crystalline forms thereof. In embodiments, the present disclosure provides crystalline form of Lanifibranor designated as Form LN1 and LN2 (defined herein).

Solid state properties of Lanifibranor and crystalline polymorphs thereof can be influenced by controlling the conditions under which Lanifibranor and crystalline polymorphs thereof are obtained in solid form.

A solid state form (or polymorph) may be referred to herein as polymorphically pure or as substantially free of any other solid state (or polymorphic) forms. As used herein in this context, the expression “substantially free of any other forms” will be understood to mean that the solid state form contains about 20% (w/w) or less, about 10% (w/w) or less, about 5% (w/w) or less, about 2% (w/w) or less, about 1% (w/w) or less, or about 0% of any other forms of the subject compound as measured, for example, by XRPD. Thus, a crystalline polymorph of Lanifibranor described herein as substantially free of any other solid state forms would be understood to contain greater than about 80% (w/w), greater than about 90% (w/w), greater than about 95% (w/w), greater than about 98% (w/w), greater than about 99% (w/w), or about 100% of the subject crystalline polymorph of Lanifibranor. In some embodiments of the disclosure, the described crystalline polymorph of Lanifibranor may contain from about 1% to about 20% (w/w), from about 5% to about 20% (w/w), or from about 5% to about 10% (w/w) of one or more other crystalline polymorph of the same Lanifibranor.

Depending on which other crystalline polymorphs a comparison is made, the crystalline polymorphs of Lanifibranor of the present disclosure may have advantageous properties selected from at least one of the following: chemical purity, flowability, solubility, dissolution rate, morphology or crystal habit, stability, such as chemical stability as well as thermal and mechanical stability with respect to polymorphic conversion, stability towards dehydration and/or storage stability, low content of residual solvent, a lower degree of hygroscopicity, flowability, and advantageous processing and handling characteristics such as compressibility and bulk density.

A solid state form, such as a crystal form or an amorphous form, may be referred to herein as being characterized by graphical data “as depicted in” or “as substantially depicted in” a Figure. Such data include, for example, powder X-ray diffractograms and solid state NMR spectra. As is well-known in the art, the graphical data potentially provides additional technical information to further define the respective solid state form (a so-called “fingerprint”) which cannot necessarily be described by reference to numerical values or peak positions alone. In any event, the skilled person will understand that such graphical representations of data may be subject to small variations, e.g., in peak relative intensities and peak positions due to certain factors such as, but not limited to, variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms. A crystal form of Lanifibranor referred to herein as being characterized by graphical data “as depicted in” or “as substantially depicted in” a Figure will thus be understood to include any crystal forms of Lanifibranor characterized with the graphical data having such small variations, as are well known to the skilled person, in comparison with the Figure.

As used herein, and unless stated otherwise, the term “anhydrous” in relation to crystalline forms of Lanifibranor, relates to a crystalline form of Lanifibranor which does not include any crystalline water (or other solvents) in a defined, stoichiometric amount within the crystal. Moreover, an “anhydrous” form would generally not contain more than 1% (w/w), of either water or organic solvents as measured for example by TGA.

The term “solvate,” as used herein and unless indicated otherwise, refers to a crystal form that incorporates a solvent in the crystal structure. When the solvent is water, the solvate is often referred to as a “hydrate.” The solvent in a solvate may be present in either a stoichiometric or in a non-stoichiometric amount.

As used herein, the term “isolated” in reference to crystalline polymorph of Lanifibranor of the present disclosure corresponds to a crystalline polymorph of Lanifibranor that is physically separated from the reaction mixture in which it is formed.

As used herein, unless stated otherwise, the XRPD measurements are taken using copper Kα radiation wavelength 1.5418 Å. XRPD peaks reported herein are measured using CuK α radiation, λ=1.5418 Å, typically at a temperature of 25±3° C.

As used herein, unless stated otherwise, 13C NMR reported herein are measured at 176.1 MHz at a magic angle spinning frequency ωₜ/2π=18 kHz, preferably at a temperature of at 293 K±3° C.

A thing, e.g., a reaction mixture, may be characterized herein as being at, or allowed to come to “room temperature” or “ambient temperature,” often abbreviated as “RT.” This means that the temperature of the thing is close to, or the same as, that of the space, e.g., the room or fume hood, in which the thing is located. Typically, room temperature is from about 20° ° C. to about 30° C., or about 22° ° C. to about 27° C., or about 25° C.

The amount of solvent employed in a chemical process, e.g., a reaction or crystallization, may be referred to herein as a number of “volumes” or “vol” or “V.” For example, a material may be referred to as being suspended in 10 volumes (or 10 vol or 10V) of a solvent. In this context, this expression would be understood to mean milliliters of the solvent per gram of the material being suspended, such that suspending a 5 grams of a material in 10 volumes of a solvent means that the solvent is used in an amount of 10 milliliters of the solvent per gram of the material that is being suspended or, in this example, 50 mL of the solvent. In another context, the term “v/v” may be used to indicate the number of volumes of a solvent that are added to a liquid mixture based on the volume of that mixture. For example, adding solvent X (1.5 v/v) to a 100 ml reaction mixture would indicate that 150 mL of solvent X was added.

A process or step may be referred to herein as being carried out “overnight.” This refers to a time interval, e.g., for the process or step, that spans the time during the night, when that process or step may not be actively observed. This time interval is from about 8 to about 20 hours, or about 10-18 hours, in some cases about 16 hours.

As used herein, the term “reduced pressure” refers to a pressure that is less than atmospheric pressure. For example, reduced pressure is about 10 mbar to about 50 mbar.

As used herein and unless indicated otherwise, the term “ambient conditions” refer to atmospheric pressure and a temperature of 22-24° C.

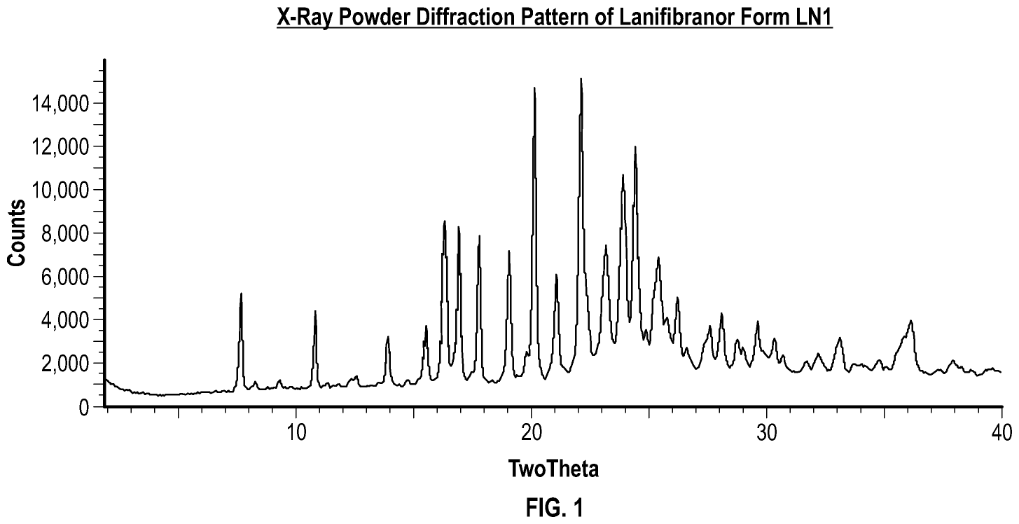

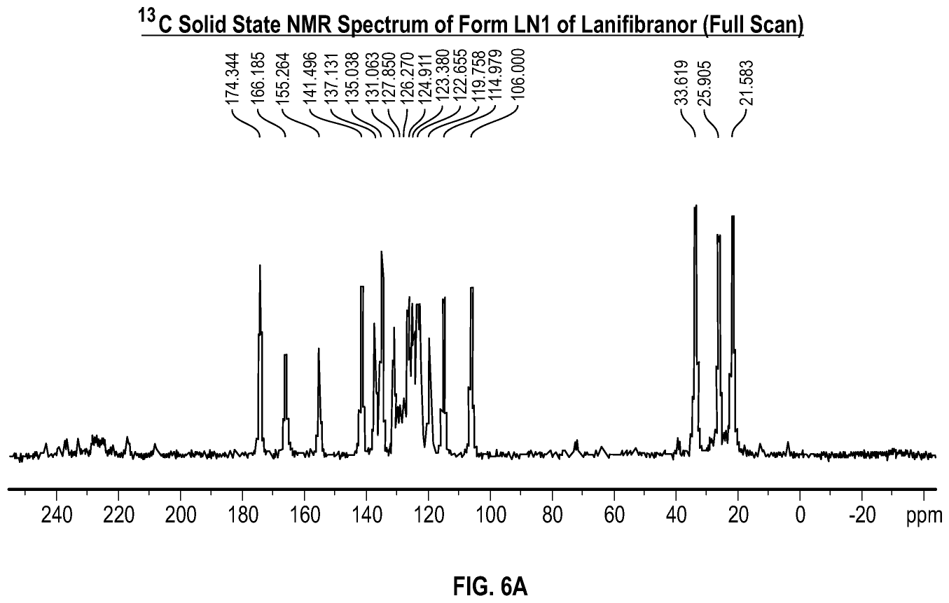

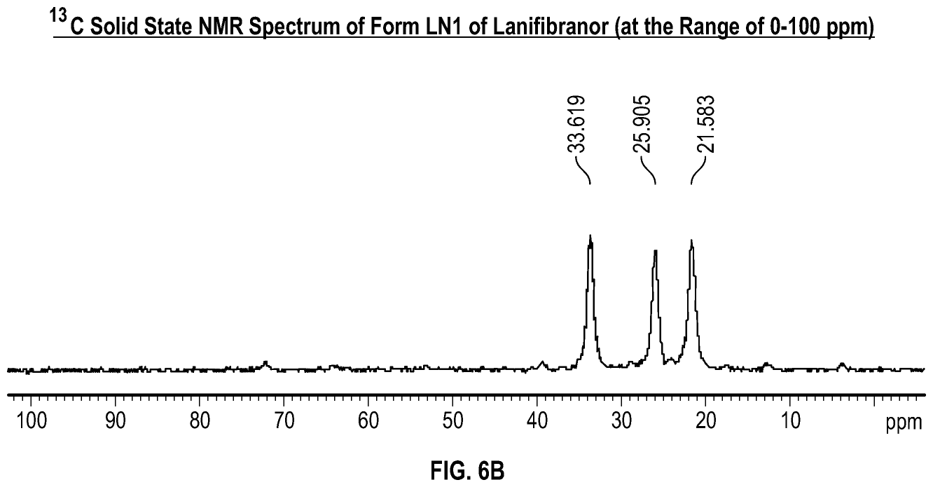

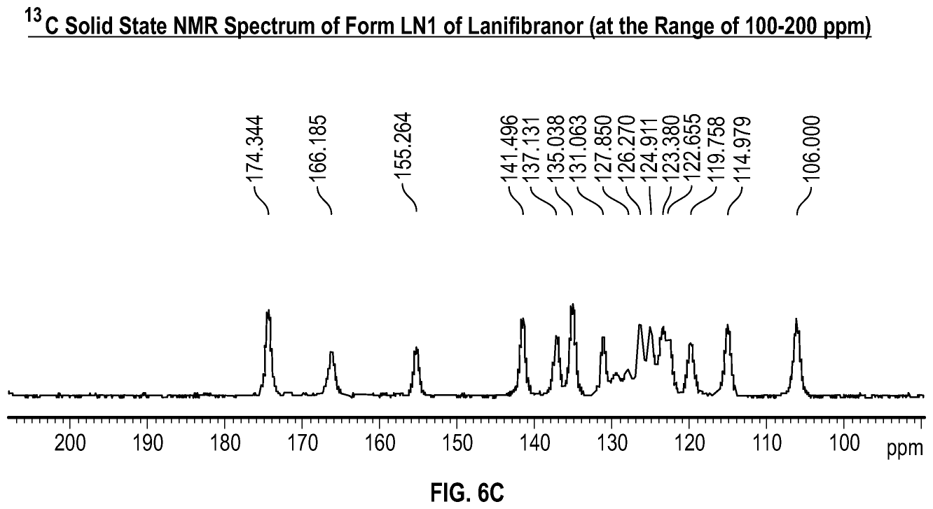

The present disclosure includes a crystalline polymorph of Lanifibranor, designated LN1. The crystalline Form LN1 of Lanifibranor may be characterized by data selected from one or more of the following: an X-ray powder diffraction pattern substantially as depicted in FIG. 1; an X-ray powder diffraction pattern having peaks at 10.8, 14.0, 19.1, 21.1 and 25.5 degrees 2-theta±0.2 degrees 2-theta; a solid state 13C NMR spectrum having peaks at 21.6, 25.9, 33.6, 115.0, 155.3 and 166.2 ppm±0.2 ppm; a solid state 13C NMR spectrum having the following chemical shift absolute differences from a reference peak at 34.5 ppm±2 ppm of 103.3, 99.0, 91.3, 9.9, 30.4 and 41.3 ppm±0.1 ppm; a solid state 13C NMR spectrum substantially as depicted in FIG. 6a, 6b or 6c; and combinations of these data.

Crystalline Form LN1 of Lanifibranor may be further characterized by an X-ray powder diffraction pattern having peaks at 10.8, 14.0, 19.1, 21.1 and 25.5 degrees 2-theta±0.2 degrees 2-theta, and also having any one, two, three, four or five additional peaks selected from 7.7, 17.8, 16.7, 23.3 and 26.3 degrees 2-theta±0.2 degrees 2-theta.

Crystalline Form LN1 of Lanifibranor may be alternatively characterized by an X-ray powder diffraction pattern having peaks at 7.7, 10.8, 14.0, 16.7, 17.8, 19.1, 21.1, 23.3, 25.5, and 26.3 degrees 2-theta±0.2 degrees 2-theta.

In one embodiment of the present disclosure, crystalline Form LN1 of Lanifibranor is isolated.

Crystalline Form LN1 of Lanifibranor may be anhydrous form.

Crystalline Form LN1 of Lanifibranor may be characterized by each of the above characteristics alone/or by all possible combinations, e.g., an XRPD pattern having peaks at 10.8, 14.0, 19.1, 21.1 and 25.5 degrees 2-theta±0.2 degrees 2-theta; an XRPD pattern as depicted in FIG. 1, and combinations thereof.

Lanifibranor Form LN1 may be prepared by a process comprising:
(a) solvent removal from a solution of Lanifibranor in a solvent comprising, consisting essentially of, or consisting of, a ketone, preferably a C₃-C₆ ketone, more preferably acetone, preferably wherein the solvent removal is conducted under reduced pressure;
(b) optionally isolating the solid; and
(c) drying the solid at a temperature of about 80° ° C. to about 180° C.

In any embodiment of this process, the ketone may be present in an amount of about 10 ml to about 50 ml, about 15 ml to about 40 ml, about 20 ml to about 30 ml, or about 25 ml, per gram of Lanifibranor. Step (a) preferably comprises solvent removal from a solution of Lanifibranor in acetone. According to any embodiment of this process, the solvent removal may be conducted at a temperature of: about 20° C. to about 60° ° C., about 25° C. to about 55° C., about 25° ° C. to about 50° C., about 30° ° C. to about 45° C., or about 35° C. to about 40° C. The solvent removal may be conducted for a suitable time period to result in a solid, preferably the solvent removal may be conducted for a suitable time period to remove substantially all of the liquid. In any embodiment of this process, the solid may be amorphous Lanifibranor. The solid may comprise about 0.5% to about 3%, about 0.8% to about 2.5%, about 1.0% to about 2.0%, or about 1.2% to about 1.6%, or about 1.4%, by weight of acetone and/or water (the water may be from atmospheric moisture).

In any embodiment of this process, the drying in step (c) may be carried out at a temperature of: about 90° ° C. to about 170° ° C., about 100° C. to about 160° ° C., about 110° C. to about 150° C., about 120° ° C. to about 140° C., about 125° C. to about 135° C., or about 130° C. According to any embodiment, the drying in step (c) may be carried out in an air tray dryer.

The present disclosure further comprises an alternative process for preparation of Form LN1 of Lanifibranor. The process may comprise crystallising Lanifibranor from a solution comprising dimethyl sulfoxide, optionally in the presence of an antisolvent.

In any embodiment of this process, the antisolvent may be a C₁-C₆ alcohol, preferably a C₁-C₄ alcohol, more preferably a C₂-C₄ alcohol, and particularly 1-butanol. In particular, the process may comprise crystallising Lanifibranor from a mixture comprising dimethyl sulfoxide and a C₁-C₆ alcohol, preferably a C₁-C₄ alcohol, more preferably a C₂-C₄ alcohol 1-butanol. According to any aspect or embodiment, the process may comprise:
(i) providing a solution of Lanifibranor in dimethylsulfoxide;
(ii) combining the solution with a C₁-C₆ alcohol, preferably a C₁-C₄ alcohol, more preferably a C₂-C₄ alcohol, and particularly 1-butanol; and
(iii) optionally isolating Lanifibranor Form LN1.

In any embodiment of the process, step (i) may comprise dissolving Lanifibranor in the dimethyl sulfoxide, preferably at a temperature of about 35° C. to about 75° C., about 45° C. to about 65° C., or about 60° C.

In any embodiment of this process, the dimethyl sulfoxide in step (i) may be used in an amount of about 1 ml to about 4 ml, about 1.5 ml to about 3.5 ml, or about 3 ml, per gram of Lanifibranor.

In any embodiment of this process, the vol/vol ratio of dimethyl sulfoxide to 1-butanol is about 1:40 to about 1:10, about 1:35 to about 1:15, about 1:30 to about 1:20, or about 1:25.

In any embodiment of this process, the solution in step (i) may be filtered.

In any embodiment of this process, step (ii) may comprise addition of the solution of Lanifibranor in the dimethyl sulfoxide to the C₁-C₆ alcohol. More particularly the C₁-C₆ alcohol in step (ii) may be precooled. Preferably, step (ii) may comprise addition of the solution in step (i) to the C₁-C₆ alcohol. In a preferred embodiment of the process, step (ii) may comprise addition of the solution of Lanifibranor in dimethyl sulfoxide to a precooled C₁-C₆ alcohol. The precooled C₁-C₆ alcohol may be at a temperature of: about −10° C. to about 15° C., about −5° C. to about 10° C., or about 0° C. to about 5° C. Preferably, step (ii) may comprise addition of the solution in step (i) to the C₁-C₆ alcohol. In any embodiment of this process the alcohol is preferably a C₁-C₄ alcohol, more preferably a C₂-C₄ alcohol, and particularly 1-butanol. The mixture in step (ii) may be maintained for period of about 15 minutes to about 36 hours, about 4 hours to about 24 hours, about 8 hours to about 20 hours, or about 12 hours to about 18 hours, preferably at a temperature of about 18° C. to about 30° C., or about 20° ° C. to about 25° C.

In any embodiment of this process, step (ii) may comprise adding Lanifibranor solution to precooled 1-butanol, wherein 1-butanol is at a temperature of about −5° ° C. to about 10° C., about −3° ° C. to about 8° C., or about 0° ° C. to about 5° C. The mixture may be maintained at temperature of about −5° C. to about 10° C., about −3° C. to about 8° ° C., or about 0° C. to about 5° C. for period of about 10 hours to 25 hours, or about 18 hours.

Step (iii) may include isolating the obtain Lanifibranor Form LN1. The isolation can be done by any suitable method, including by centrifuging, decantation, or preferably by filtration. Following isolation, the Lanifibranor Form LN1 may be washed, for example with the alcohol, and optionally dried. The drying may be carried out under reduced pressure, preferably at temperature of about 30° ° C. to about 40° C., or about 20° C. to about 25° C., for a suitable period of time, for example about 5 minutes to 30 minutes, or about 10 minutes to about 15 minutes.

The processes for preparing Lanifibranor Form LN1 as described in any aspect or embodiment of the present disclosure may further comprise combining the Form LN1 with at least one pharmaceutically acceptable excipient to prepare a pharmaceutical composition.

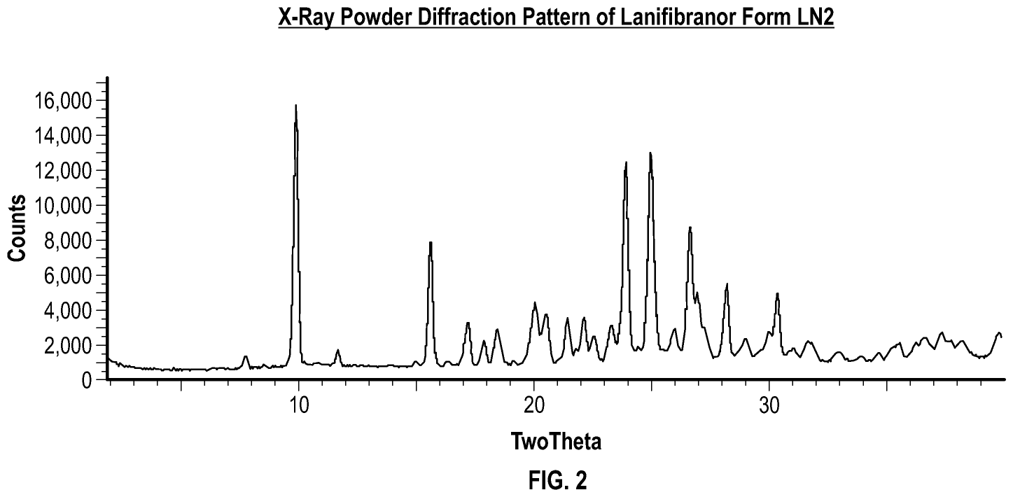

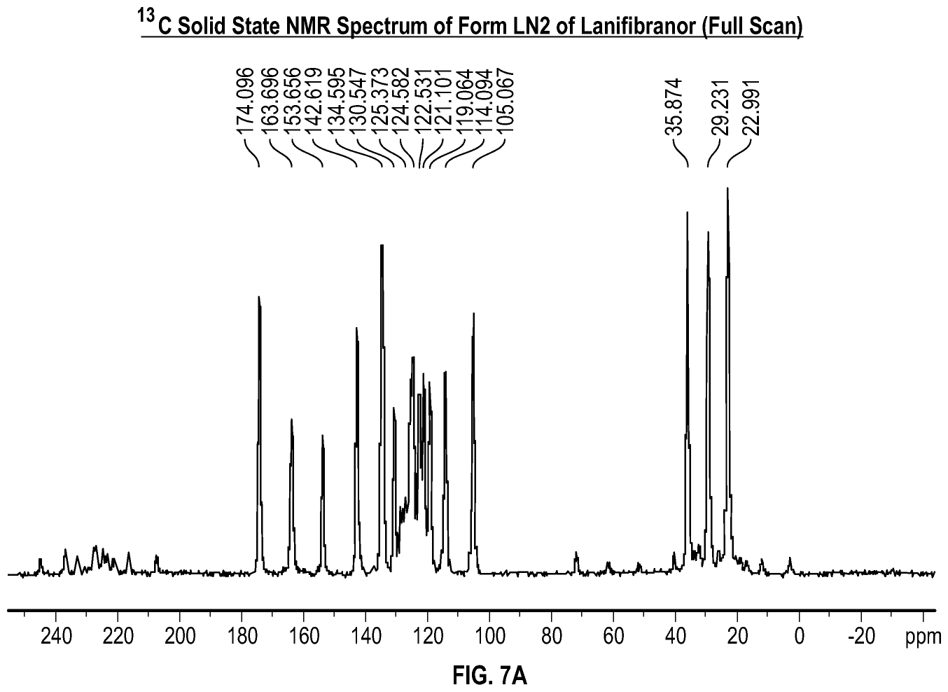

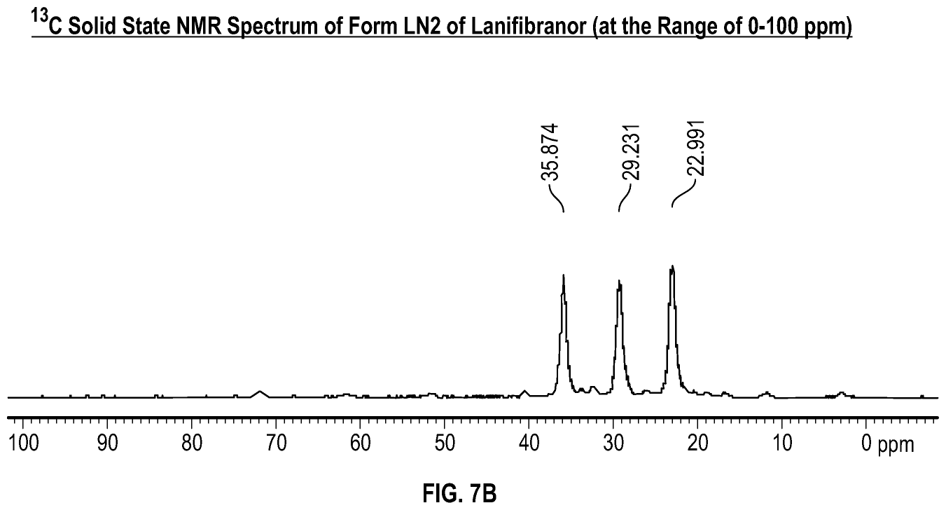

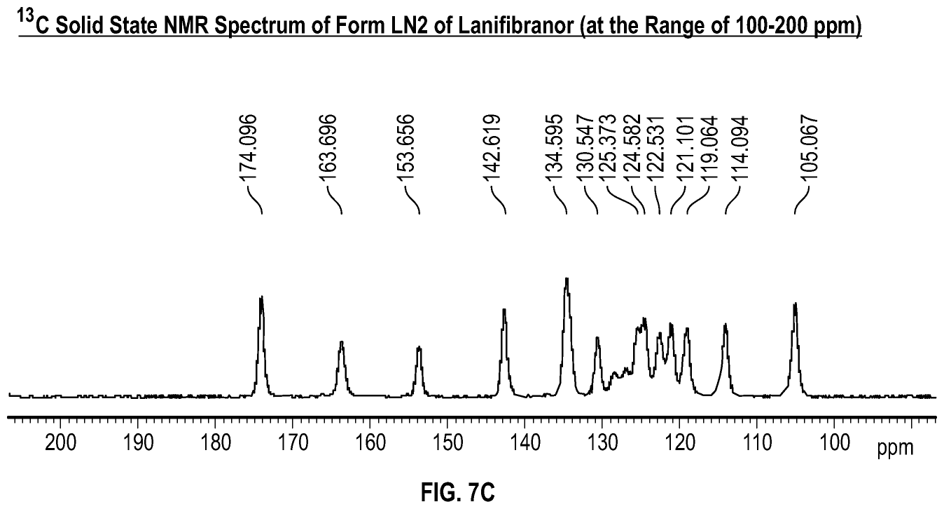

The present disclosure includes a crystalline polymorph of Lanifibranor, designated LN2. The crystalline Form LN2 of Lanifibranor may be characterized by data selected from one or more of the following: an X-ray powder diffraction pattern substantially as depicted in FIG. 2; an X-ray powder diffraction pattern having peaks at 9.9, 17.2, 18.5, 25.0 and 26.7 degrees 2-theta±0.2 degrees 2-theta; a solid state 13C NMR spectrum having peaks at 29.2, 105.1, 134.6, 142.6, 153.7 and 163.7 ppm±0.2 ppm; a solid state 13C NMR spectrum having the following chemical shift absolute differences from a reference peak at 124.6 ppm±2 ppm of 95.4, 19.5, 10.0, 18.0, 29.1 and 39.1 ppm±0.1 ppm; a solid state 13C NMR spectrum substantially as depicted in FIG. 7a, 7b or 7c; and combinations of these data.

Crystalline Form LN2 of Lanifibranor may be further characterized by an X-ray powder diffraction pattern having peaks at 9.9, 17.2, 18.5, 25.0 and 26.7 degrees 2-theta±0.2 degrees 2-theta, and also having any one, two, three, or four additional peaks selected from 15.6, 20.5, 22.1 and 23.9 degrees 2-theta±0.2 degrees 2-theta.

Crystalline Form LN2 of Lanifibranor may be alternatively characterized by an X-ray powder diffraction pattern having peaks at 9.9, 15.6, 17.2, 18.5, 20.5, 22.1, 23.9, 25.0 and 26.7 degrees 2-theta±0.2 degrees 2-theta.

In one embodiment of the present disclosure, crystalline Form LN2 of Lanifibranor is isolated.

Crystalline Form LN2 of Lanifibranor may be anhydrous form.

Crystalline Form LN2 of Lanifibranor may be characterized by each of the above characteristics alone/or by all possible combinations, e.g., an XRPD pattern having peaks at 9.9, 17.2, 18.5, 25.0 and 26.7 degrees 2-theta±0.2 degrees 2-theta; an XRPD pattern as depicted in FIG. 2, and combinations thereof.

The present disclosure further comprises a process for preparation of Form LN2 of Lanifibranor. The process for preparing Form LN2 of Lanifibranor may comprise crystallization from a solution of Lanifibranor in a C₃-C₆ ketone, preferably acetone. According to any aspect or embodiment, the process may comprise:
(i) providing a solution of Lanifibranor in a C₃-C₆ ketone, preferably acetone;
(ii) cooling the solution; and
(iii) optionally isolating Form LN2 of Lanifibranor.

According to any embodiment of the process, step (i) may comprise dissolving Lanifibranor in the ketone, preferably at a temperature of about 55° C. to about 65° C., or about 45° C. to about 55° C., or about 35° C. to about 40° C.

In any embodiment of this process, the ketone in step (i) is typically used in an amount of about 25 ml to about 45 ml, about 15 ml to about 35 ml, or about 30 ml, per gram of Lanifibranor. The solution may optionally be filtered.

In any embodiment of this process, step (ii) may comprise cooling the solution, typically to a temperature of about −5° C. to about 10° C., about −3° C. to about 8° C., or about 0° C. to about 5° C. The solution may be cooled over a period of time to obtain crystalline Lanifibranor Form LN2. The cooling may be for a period of about 10 minutes to about 80 minutes, about 15 minutes to about 60 minutes, about 20 minutes to about 40 minutes, or about 30 minutes. The cooling may be done whilst stirring. After cooling, the mixture may be stirred, preferably at the cooling temperature, for a period of about 0.5 hours to about 3 hours, about 0.75 hours to about 2 hours, or about 1 hour.

The present disclosure further comprises an alternative process for preparation of Form LN2 of Lanifibranor. The process may comprise crystallising Lanifibranor from a solution comprising a C₁-C₃ chlorinated hydrocarbon, preferably dichloromethane, optionally in the presence of an antisolvent. The antisolvent may be a C₆-C₁₂ hydrocarbon, preferably a C₈-C₁₂ hydrocarbon. The hydrocarbon may be aliphatic, cyclic, or aromatic. Preferably the hydrocarbon is an aliphatic C₆-C₁₂ hydrocarbon, more preferably an aliphatic C₈-C₁₀ hydrocarbon, and particularly decane. In particular, the process may comprise crystallising Lanifibranor from a solution comprising dichloromethane and decane. According to any aspect or embodiment, the process may comprise:
(iv) providing a solution of Lanifibranor in a C₁-C₃ chlorinated hydrocarbon, preferably dichloromethane;
(v) combining the solution with a C₆-C₁₂ hydrocarbon, preferably a C₈-C₁₂ hydrocarbon, and particularly decane; and
(vi) optionally isolating Lanifibranor Form LN2.

In any embodiment of the process, step (i) may comprise dissolving Lanifibranor in the chlorinated hydrocarbon, preferably at a temperature of about 25° C. to about 65° C., about 35° C. to about 60° C., or about 40° C. to about 50° C.

In any embodiment of this process, the chlorinated hydrocarbon (preferably dichloromethane) in step (i) is typically used in an amount of about 60 ml to about 90 ml, about 70 ml to about 85 ml, or about 80 ml, per gram of Lanifibranor.

In any embodiment of this process, the vol/vol ratio of chlorinated hydrocarbon (preferably dichloromethane) to hydrocarbon (preferably decane) is about 1:0.5 to about 1:4, about 1:0.8 to about 1:3.5, about 1:1 to about 1:3, about 1:1 to about 1:2 or about 1:1 to about 1:1.5, or about 1:1.25.

In any embodiment of this process, the solution in step (i) may be filtered.

In any embodiment of this process, step (ii) may comprise addition of the solution if Lanifibranor in the chlorinated hydrocarbon to the hydrocarbon. The hydrocarbon may comprise seeds of Lanifibranor Form LN2. Particularly, step (ii) may comprise dropwise addition of the solution in step (i) to the hydrocarbon. More particularly the hydrocarbon in step (ii) may be precooled. In a preferred embodiment of the process, step (ii) may comprise addition of the solution of Lanifibranor in the chlorinated hydrocarbon (preferably dropwise) to a precooled hydrocarbon. The precooled hydrocarbon may be at a temperature of: about −10° ° C. to about 15° C., −5° ° C. to about 10° C., or 0° ° C. to about 5° C. The mixture in step (ii) may be maintained for period of about 20 minutes to about 1 hour, or about 30 minutes.

In any embodiment of this process, seeds of Form LN2 may be added. The seeds may be added to the hydrocarbon. Typically, the seeds may be used in an amount of: about 1% to about 15%, or about 5% to about 10% (w/w)

In any embodiment of this process, step (ii) may comprise adding Lanifibranor solution to precooled hydrocarbon (preferably decane) which optionally contains Lanifibranor Form LN2 seeds, wherein the hydrocarbon is at a temperature of about −5° C. to about 10° C., about −3° C. to about 8° C., or about 0° ° C. to about 5° ° C. The addition is preferably dropwise, optionally for period of about 15 minutes to 20 minutes. The mixture may be maintained at temperature of about −5° C. to about 10° C., about −3° C. to about 8° C., or about 0° C. to about 5° C. The mixture may be maintained, preferably at this temperature, for period of about 10 hours to 25 hours, or about 18 hours.

Step (iii) may include isolating the obtain Lanifibranor Form LT2, for example by vacuum filtration. Following isolation, the Lanifibranor Form LT2 may be dried. Lanifibranor Form LT2 may be dried under reduced pressure, optionally for period of about 5 minutes to 30 minutes, or about 15 minutes to about 20 minutes.

The processes for preparing Lanifibranor Form LT2 as described in any aspect or embodiment of the present disclosure may further comprise combining the Form LT2 with at least one pharmaceutically acceptable excipient to prepare a pharmaceutical composition.

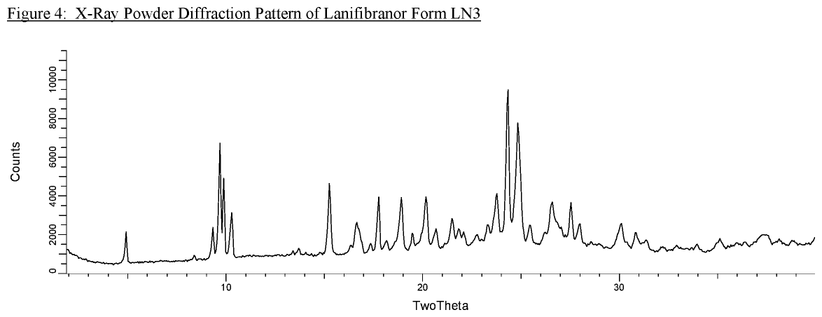

The present disclosure includes a crystalline polymorph of Lanifibranor, designated LN3. The crystalline Form LN3 of Lanifibranor may be characterized by data selected from one or more of the following: an X-ray powder diffraction pattern substantially as depicted in FIG. 4; an X-ray powder diffraction pattern having peaks at 4.9, 15.3, 18.9, 20.2 and 24.3 degrees 2-theta±0.2 degrees 2-theta; and combinations of these data.

Crystalline Form LN3 of Lanifibranor may be further characterized by an X-ray powder diffraction pattern having peaks at 4.9, 15.3, 18.9, 20.2 and 24.3 degrees 2-theta±0.2 degrees 2-theta, and also having any one, two, three, four or five additional peaks selected from 9.7, 10.2, 17.8, 27.5 and 30.1 degrees 2-theta±0.2 degrees 2-theta.

In one embodiment of the present disclosure, crystalline Form LN3 of Lanifibranor is isolated.

Crystalline Form LN3 of Lanifibranor may be 1,3-dioxolane solvate.

Crystalline Form LN3 of Lanifibranor may be characterized by each of the above characteristics alone/or by all possible combinations, e.g., an XRPD pattern having peaks at 4.9, 15.3, 18.9, 20.2 and 24.3 degrees 2-theta±0.2 degrees 2-theta; an XRPD pattern as depicted in FIG. 4, and combinations thereof.

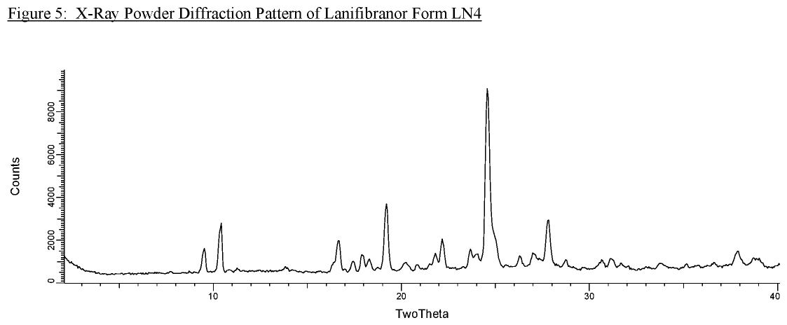

The present disclosure includes a crystalline polymorph of Lanifibranor, designated LN4. The crystalline Form LN4 of Lanifibranor may be characterized by data selected from one or more of the following: an X-ray powder diffraction pattern substantially as depicted in FIG. 5; an X-ray powder diffraction pattern having peaks at 9.5, 10.4, 22.2, 24.6 and 27.7 degrees 2-theta±0.2 degrees 2-theta; and combinations of these data.

Crystalline Form LN4 of Lanifibranor may be further characterized by an X-ray powder diffraction pattern having peaks at 9.5, 10.4, 22.2, 24.6 and 27.7 degrees 2-theta±0.2 degrees 2-theta, and also having any one, two, three or four additional peaks selected from 16.7, 19.2, 26.3 and 31.2 degrees 2-theta±0.2 degrees 2-theta.

In one embodiment of the present disclosure, crystalline Form LN4 of Lanifibranor is isolated.

Crystalline Form LN4 of Lanifibranor may be formamide solvate.

Crystalline Form LN4 of Lanifibranor may be characterized by each of the above characteristics alone/or by all possible combinations, e.g., an XRPD pattern having peaks at 9.5, 10.4, 22.2, 24.6 and 27.7 degrees 2-theta±0.2 degrees 2-theta; an XRPD pattern as depicted in FIG. 5, and combinations thereof.

According to any aspect or embodiment of the present disclosure, any of the solid state forms of Lanifibranor described herein may be polymorphically pure or may be substantially free of any other solid state forms of Lanifibranor. In any aspect or embodiment of the present disclosure, any of the solid state forms of Lanifibranor may contain: about 20% (w/w) or less, about 10% (w/w) or less, about 5% (w/w) or less, about 2% (w/w) or less, about 1% (w/w) or less, about 0.5% (w/w) or less, about 0.2% (w/w) or less, about 0.1% (w/w) or less, or about 0%, of any other solid state forms of Lanifibranor, preferably as measured by XRPD. Thus, any of the disclosed crystalline forms of Lanifibranor described herein may be substantially free of any other solid state forms of Lanifibranor, and may contain greater than about 80% (w/w), greater than about 90% (w/w), greater than about 95% (w/w), greater than about 98% (w/w), greater than about 99% (w/w), or about 100% of the subject solid state form of Lanifibranor.

The above crystalline polymorphs can be used to prepare other crystalline polymorphs of Lanifibranor, Lanifibranor salts and their solid state forms.

The present disclosure encompasses a process for preparing other solid state forms of Lanifibranor, Lanifibranor salts and their solid state forms thereof. The process includes preparing any one of the crystalline polymorph of Lanifibranor by the processes of the present disclosure.

The present disclosure provides the above described crystalline polymorphs of Lanifibranor for use in the preparation of pharmaceutical compositions comprising Lanifibranor and/or crystalline polymorphs thereof.

The present disclosure also encompasses the use of crystalline polymorphs of Lanifibranor of the present disclosure for the preparation of pharmaceutical compositions of crystalline polymorph Lanifibranor and/or crystalline polymorphs thereof.

The present disclosure includes processes for preparing the above mentioned pharmaceutical compositions. The processes includes combining any one or a combination of the crystalline polymorphs of Lanifibranor of the present disclosure with at least one pharmaceutically acceptable excipient.

Pharmaceutical combinations or formulations of the present disclosure contain any one or a combination of the solid state forms of Lanifibranor of the present disclosure. In addition to the active ingredient, the pharmaceutical formulations of the present disclosure can contain one or more excipients. Excipients are added to the formulation for a variety of purposes.

Diluents increase the bulk of a solid pharmaceutical composition, and can make a pharmaceutical dosage form containing the composition easier for the patient and caregiver to handle. Diluents for solid compositions include, for example, microcrystalline cellulose (e.g. Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol, and talc.

Solid pharmaceutical compositions that are compacted into a dosage form, such as a tablet, can include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate, and starch.

The dissolution rate of a compacted solid pharmaceutical composition in the patient's stomach can be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®), and starch.

Glidants can be added to improve the flowability of a non-compacted solid composition and to improve the accuracy of dosing. Excipients that can function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that can be included in the composition of the present disclosure include maltol, vanillin, ethyl vanillin, menthol, citric acid, fumaric acid, ethyl maltol, and tartaric acid.

Solid and liquid compositions can also be dyed using any pharmaceutically acceptable colorant to improve their appearance and/or facilitate patient identification of the product and unit dosage level.

In liquid pharmaceutical compositions of the present invention, Lanifibranor and any other solid excipients can be dissolved or suspended in a liquid carrier such as water, vegetable oil, alcohol, polyethylene glycol, propylene glycol, or glycerin.

Liquid pharmaceutical compositions can contain emulsifying agents to disperse uniformly throughout the composition an active ingredient or other excipient that is not soluble in the liquid carrier. Emulsifying agents that can be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the present invention can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, xanthan gum and combinations thereof.

Sweetening agents such as sorbitol, saccharin, sodium saccharin, sucrose, aspartame, fructose, mannitol, and invert sugar can be added to improve the taste.

Preservatives and chelating agents such as alcohol, sodium benzoate, butylated hydroxyl toluene, butylated hydroxyanisole, and ethylenediamine tetraacetic acid can be added at levels safe for ingestion to improve storage stability.

According to the present disclosure, a liquid composition can also contain a buffer such as gluconic acid, lactic acid, citric acid, or acetic acid, sodium gluconate, sodium lactate, sodium citrate, or sodium acetate. Selection of excipients and the amounts used can be readily determined by the formulation scientist based upon experience and consideration of standard procedures and reference works in the field.

The solid compositions of the present disclosure include powders, granulates, aggregates, and compacted compositions. The dosages include dosages suitable for oral, buccal, rectal, parenteral (including subcutaneous, intramuscular, and intravenous), inhalant, and ophthalmic administration. Although the most suitable administration in any given case will depend on the nature and severity of the condition being treated, in embodiments the route of administration is oral. The dosages can be conveniently presented in unit dosage form and prepared by any of the methods well-known in the pharmaceutical arts.

Dosage forms include solid dosage forms like tablets, powders, capsules, suppositories, sachets, troches, and lozenges, as well as liquid syrups, suspensions, and elixirs.

The dosage form of the present disclosure can be a capsule containing the composition, such as a powdered or granulated solid composition of the disclosure, within either a hard or soft shell. The shell can be made from gelatin and optionally contain a plasticizer such as glycerin and/or sorbitol, an opacifying agent and/or colorant.

The active ingredient and excipients can be formulated into compositions and dosage forms according to methods known in the art.

A composition for tableting or capsule filling can be prepared by wet granulation. In wet granulation, some or all of the active ingredients and excipients in powder form are blended and then further mixed in the presence of a liquid, typically water, that causes the powders to clump into granules. The granulate is screened and/or milled, dried, and then screened and/or milled to the desired particle size. The granulate can then be tableted, or other excipients can be added prior to tableting, such as a glidant and/or a lubricant.

A tableting composition can be prepared conventionally by dry blending. For example, the blended composition of the actives and excipients can be compacted into a slug or a sheet and then comminuted into compacted granules. The compacted granules can subsequently be compressed into a tablet.

As an alternative to dry granulation, a blended composition can be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate, and colloidal silica. The proper use of these and other excipients in direct compression tableting is known to those in the art with experience and skill in particular formulation challenges of direct compression tableting.

A capsule filling of the present disclosure can include any of the aforementioned blends and granulates that were described with reference to tableting, but they are not subjected to a final tableting step.

A pharmaceutical formulation of Lanifibranor can be administered. Lanifibranor may be formulated for administration to a mammal, in embodiments to a human, by injection. Lanifibranor can be formulated, for example, as a viscous liquid solution or suspension, such as a clear solution, for injection. The formulation can contain one or more solvents. A suitable solvent can be selected by considering the solvent's physical and chemical stability at various pH levels, viscosity (which would allow for syringeability), fluidity, boiling point, miscibility, and purity. Suitable solvents include alcohol USP, benzyl alcohol NF, benzyl benzoate USP, and Castor oil USP. Additional substances can be added to the formulation such as buffers, solubilizers, and antioxidants, among others. Ansel et al., Pharmaceutical Dosage Forms and Drug Delivery Systems, 7th ed.

The crystalline polymorphs of Lanifibranor and the pharmaceutical compositions and/or formulations of Lanifibranor of the present disclosure can be used as medicaments, in embodiments in the treatment of non-alcoholic steatohepatitis (NASH), non-alcoholic fatty liver disease, type 2 diabetes, fibrosis and systemic sclerosis.

The present disclosure also provides methods of treating non-alcoholic steatohepatitis (NASH), non-alcoholic fatty liver disease, type 2 diabetes, fibrosis and systemic sclerosis by administering a therapeutically effective amount of any one or a combination of the crystalline polymorphs of Lanifibranor of the present disclosure, or at least one of the above pharmaceutical compositions and/or formulations, to a subject in need of the treatment.

Having thus described the disclosure with reference to particular preferred embodiments and illustrative examples, those in the art can appreciate modifications to the disclosure as described and illustrated that do not depart from the spirit and scope of the disclosure as disclosed in the specification. The Examples are set forth to aid in understanding the disclosure but are not intended to, and should not be construed to limit its scope in any way.

### Powder X-Ray Diffraction (“XRPD”) Method

X-ray diffraction was performed on X-Ray powder diffractometer: Bruker D8 Advance; CuK_ radiation (λ=1.5418 Å); Lynx eye detector; laboratory temperature 22-25° C.; PMMA specimen holder ring. Prior to analysis, the samples were gently ground by means of mortar and pestle in order to obtain a fine powder. The ground sample was adjusted into a cavity of the sample holder and the surface of the sample was smoothed by means of a cover glass.

Measurement parameters:
Scan range: 2-40 degrees 2-theta;
Scan mode: continuous;
Step size: 0.05 degrees;
Time per step: 0.5 s;
Sample spin: 30 rpm;
Sample holder: PMMA specimen holder ring with silicon low background holder.

All X-Ray Powder Diffraction peak values are calibrated with regard to standard silicon spiking in the sample.

### SSNMR Method

Solid-state NMR spectra were measured at 11.7 T using a Bruker Avance III HD 500 US/WB NMR spectrometer (Karlsruhe, Germany, 2013) with 3.2 mm probehead. The 13C CP/MAS NMR spectra employing cross-polarization were acquired using the standard pulse scheme at spinning frequency of 18 kHz and a room temperature (300 K). The recycle delay was 8 s and the cross-polarization contact time was 2 ms. The 13C scale was referenced to α-glycine (176.03 ppm for 13C). Frictional heating of the spinning samples was offset by active cooling, and the temperature calibration was performed with Pb(NO3)2. The NMR spectrometer was completely calibrated and all experimental parameters were carefully optimized prior the investigation. Magic angle was set using KBr during standard optimization procedure and homogeneity of magnetic field was optimized using adamantane sample (resulting line-width at half-height Δv1/2 was less than 3.5 Hz at 250 ms of acquisition time).

### EXAMPLES

### Preparation of Starting Materials

Lanifibranor can be prepared according to methods known from the literature, for example U.S. Pat. No. 7,795,297.

### Example 1: Preparation of Lanifibranor Form LN1

Lanifibranor Amorphous (0.1 grams) was dried in Air tray dryer (ATD) at temperature of about 130° C. for period of about 1 hour, cooled down to temperature of about 25° ° C. to about 30° C. and was analyzed by XRPD. Lanifibranor Form LN1 was obtained. An XRPD pattern is shown in FIG. 1.

### Example 2: Preparation of Lanifibranor Form LN2

Lanifibranor (1.0 gram) was dissolved in acetone (30 ml) at temperature of about 35° C. to about 40° C. and was filtered through 0.45 micron filter. The clear solution was slowly cool down to temperature of about 0° ° C. to about 5° C. and stirred for 1 hour at temperature of about 0° C. to about 5° C. The obtained solid was analyzed by XRPD. Lanifibranor form LN2 was obtained. An XRPD pattern is shown in FIG. 2.

### Example 3: Preparation of Lanifibranor Amorphous Form

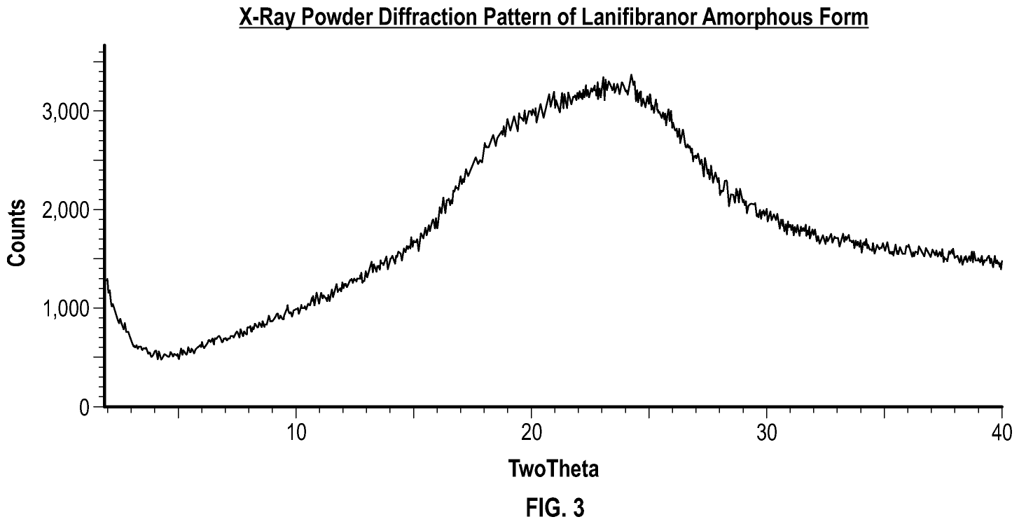

Lanifibranor (0.2 grams) was dissolved in methanol (5 mL) and acetone (15 ml) at temperature of about 25° C. to about 30° ° C. The solution was filtered through 0.45-micron filter. The clear solution was subjected to distillation under reduced pressure on rotary evaporator at temperature of about 35° C. to about 40° C. for period of about 5 minutes to about 10 minutes. The obtained solid mass was dissolved in acetone (5 ml) and the clear solution was distilled under reduced pressure for period of about 15 minutes to about 30 minutes. The obtained white solid was isolated and was analyzed by XRPD. Amorphous form of Lanifibranor was obtained. An XRPD pattern is shown in FIG. 3.

### Example 4: Preparation of Lanifibranor Form LN3

Lanifibranor (0.05 grams) was dissolved in 1,3-dioxolane (2 ml) at temperature of about 25° C. to about 30° C. The solution was filtered through 0.45-micron filter. The clear solution was subjected to distillation under reduced pressure at temperature of about 60° C. for period of about 1 hour and was cooled down to temperature of about 25° ° C. to about 30° C. and was analyzed by XRD. The obtained solid was analyzed by XRPD. Lanifibranor form LN3 was obtained. An XRPD pattern is shown in FIG. 4.

### Example 5: Preparation of Lanifibranor Form LN4

Lanifibranor (Form LN2, 0.04 grams) was slurried in formamide (1 ml) at temperature of about 0° C. to about 5° C. The slurry mass was maintained at temperature of about 0° ° C. to about 5° C. under stirring for period of about 1 day. The slurry mas was brought to temperature of about 25° C. and filtered and suck dried for period of about 15 minutes to about 20 minutes. The obtained solid was analyzed by XRPD. Lanifibranor form LN4 was obtained. An XRPD pattern is shown in FIG. 5.

### Example 6: Preparation of Lanifibranor Form LN2

Lanifibranor (0.1 grams) was dissolved in dichloromethane (8 ml) at temperature of about 40° C. and was filtered through 0.45 micron filter. In another flask, decane (10 ml) was charged and cooled down to temperature of about 0° ° C. to about 5° C., maintained at this temperature for 30 minutes and seeds of Form LN2 (about 5-10%) were added. The above Lanifibranor solution was added dropwise into precooled decane solution in the period of about 15 minutes to about 20 minutes and maintained for period of about 2 hours at temperature of about 0° C. and then filtered and suck dried for period of about 15 minutes to about 20 minutes. The obtained solid was analyzed by XRPD. Lanifibranor form LN2 was obtained.

### Example 7: Preparation of Lanifibranor Form LN2

Lanifibranor (1.0 gram) was dissolved in dichloromethane (80 ml) at temperature of about 50° C. and was filtered through 0.45 micron filter. In another flask decane (100 ml) was charged and cooled down to temperature of about 0° ° C. to about 5° C., maintained at this temperature for 30 minutes and seeds of Form LN2 (about 5-10%) were added. above Lanifibranor solution was added dropwise into precooled decane solution in period of about 15 minutes to 20 minutes and maintained for period of about 18 hours at temperature of period of 0° ° C. to about 5° C. and then filtered and suck dried for period of about 15 minutes to about 20 minutes. The obtained solid was analyzed by XRPD. Lanifibranor form LN2 was obtained.

### Example 8: Preparation of Lanifibranor Amorphous Form

Lanifibranor (0.2 grams) was dissolved in methanol (5 mL) and acetone (15 ml) at temperature of about 25° C. to about 30° C. The solution was filtered through 0.45-micron filter. The clear solution was subjected to distillation under reduced pressure on rotary evaporator at temperature of about 35° C. to about 40° ° C. for period of about 5 minutes to about 10 minutes. The obtained solid mass was dissolved in acetone (5 ml) and the clear solution was completely distilled under reduced pressure for period of about 15 minutes to about 30 minutes at temperature of about 30° C. to about 40° C. After completed distillation, the white solid was isolated and was analyzed by XRPD. Amorphous form of Lanifibranor was obtained.

### Example 9: Preparation of Lanifibranor Form LN1

Lanifibranor (1.0 gram) was dissolved in dimethyl sulfoxide (DMSO) (3 ml) at temperature of about 60° C. and filtered through a 0.45 micron filter to prepare a stock solution. The hot stock solution (about 0.2 ml) was added into precooled 1-butanol (5 ml) (about 0° C. to about 5° C.), stirred at temperature of about 0° ° C. to about 5° C. for period of about 18 hours, temperature was raised to about 20° ° C. to about 25° C., filtered and washed with 1-butanol (3 ml) three times and dried under vacuum at temperature of about 20° ° C. to about 25° C. for period of about 10 to 15 minutes. The obtained solid was analyzed by XRPD. Lanifibranor form LN1 was obtained.

1. Crystalline Form LN1 of Lanifibranor characterized by data selected from one or more of the following:
a) an XRPD pattern having peaks at 10.8, 14.0, 19.1, 21.1 and 25.5 degrees 2-theta±0.2 degrees 2-theta;
b) an XRPD pattern as depicted in FIG. 1;
c) a solid state 13C NMR spectrum having peaks at 21.6, 25.9, 33.6, 115.0, 155.3 and 166.2 ppm±0.2 ppm;
d) a solid state 13C NMR spectrum having the following chemical shift absolute differences from a reference peak at 34.5 ppm±2 ppm of 103.3, 99.0, 91.3, 9.9, 30.4 and 41.3 ppm±0.1 ppm;
e) a solid-state 13C NMR spectrum substantially as depicted in FIG. 6a, 6b or 6c; and
f) combinations of two or more of: a, b, c, d, and e.

2. Crystalline Form LN1 of Lanifibranor according to claim 1, which is characterized by an XRPD pattern having peaks at 10.8, 14.0, 19.1, 21.1 and 25.5 degrees 2-theta±0.2 degrees 2-theta, and also having one, two, three, four, or five additional peaks selected from 7.7, 17.8, 16.7, 23.3 and 26.3 degrees two theta±0.2 degrees two theta.

3. Crystalline Form LN1 of Lanifibranor according to claim 1, which is characterized by an XRPD pattern having peaks at: 7.7, 10.8, 14.0, 16.7, 17.8, 19.1, 21.1, 23.3, 25.5, and 26.3 degrees 2-theta±0.2 degrees 2-theta.

4. Crystalline Form LN1 of Lanifibranor according to claim 1, wherein said crystalline form is an anhydrous form.

5. Crystalline Form LN2 of Lanifibranor characterized by data selected from one or more of the following:
a) an XRPD pattern having peaks at 9.9, 17.2, 18.5, 25.0 and 26.7 degrees 2-theta±0.2 degrees 2-theta;
b) an XRPD pattern as depicted in FIG. 2;
c) a solid state 13C NMR spectrum having peaks at 29.2, 105.1, 134.6, 142.6, 153.7 and 163.7 ppm±0.2 ppm;
d) a solid state 13C NMR spectrum having the following chemical shift absolute differences from a reference peak at 124.6 ppm±2 ppm of 95.4, 19.5, 10.0, 18.0, 29.1 and 39.1 ppm±0.1 ppm;
e) a solid-state 13C NMR spectrum substantially as depicted in FIG. 7a, 7b or 7c; and
f) combinations of two or more of: a, b, c, d, and e.

6. Crystalline Form LN2 of Lanifibranor according to claim 5, which is characterized by an XRPD pattern having peaks at 9.9, 17.2, 18.5, 25.0 and 26.7 degrees 2-theta±0.2 degrees 2-theta, and also having one, two, three, or four additional peaks selected from 15.6, 20.5, 22.1 and 23.9 degrees two theta±0.2 degrees two theta.

7. Crystalline Form LN2 of Lanifibranor according to claim 5, which is characterized by an XRPD pattern having peaks at: 9.9, 15.6, 17.2, 18.5, 20.5, 22.1, 23.9, 25.0, and 26.7 degrees 2-theta±0.2 degrees 2-theta.

8. Crystalline Form LN2 of Lanifibranor according to claim 5, wherein said crystalline form is an anhydrous form.

9. Crystalline Lanifibranor according to claim 1, which contains no more than about 20% of any other crystalline forms of Lanifibranor.

10. Crystalline Lanifibranor according to claim 1, which contains no more than about 20% of amorphous Lanifibranor.

11. A pharmaceutical composition comprising a crystalline form of Lanifibranor according to claim 1.

12. (canceled)

13. A pharmaceutical formulation comprising a crystalline form of Lanifibranor according to claim 1, with at least one pharmaceutically acceptable excipient.

14. A process for preparing a pharmaceutical formulation, comprising combining a crystalline form of Lanifibranor according to claim 1 with at least one pharmaceutically acceptable excipient.

15. A medicament comprising the Crystalline form of Lanifibranor according to claim 1.

16. (canceled)

17. A method of treating non-alcoholic steatohepatitis (NASH), non-alcoholic fatty liver disease, type 2 diabetes, fibrosis or systemic sclerosis, comprising administering a therapeutically effective amount of a crystalline form of Lanifibranor according to claim 1, to a subject in need of treatment.

18. (canceled)

19. (canceled)

20. Crystalline Lanifibranor according to claim 5, which contains no more than about 20% of any other crystalline forms of Lanifibranor.

21. Crystalline Lanifibranor according to claim 5, which contains no more than about 20% of amorphous Lanifibranor.

22. A pharmaceutical composition comprising a crystalline form of Lanifibranor according to claim 5.

23. A pharmaceutical formulation comprising a crystalline form of Lanifibranor according to claim 5 with at least one pharmaceutically acceptable excipient.

24. A process for preparing a pharmaceutical formulation comprising combining a crystalline form of Lanifibranor according to claim 5 with at least one pharmaceutically acceptable excipient.

25. A method of treating non-alcoholic steatohepatitis (NASH), non-alcoholic fatty liver disease, type 2 diabetes, fibrosis or systemic sclerosis, comprising administering a therapeutically effective amount of a crystalline form of Lanifibranor according to claim 5 to a subject in need of treatment.
